(19) United States
(12) Patent Application Publication
(10) Pub. No.: US 2003/0113302 A1
(43) Pub. Date: Jun. 19, 2003

(54) Use of recipient endothelial cells for enhanced vascularization of tissue and tissue-engineered construct transplants

(76) Inventors: Elena Revazova, Los Angeles, CA (US); Jeff Sebastian, Los Angeles, CA (US)

Correspondence Address: Laurie A. Axford, Morrison & Foerster LLP, Suite 500, 3811 Valley Centre Drive, San Diego, CA, 92130, US

(21) Appl. No.: 10/233,005

(22) Filed: Aug. 30, 2002

Related U.S. Application Data
(60) Provisional application No. 60/316,441, filed on Aug. 31, 2001.

(51) Int. Cl. A61K 48/00; C12N 5/08

(52) U.S. Cl. 424/93.21; 424/93.7; 435/366

(57) ABSTRACT

The success of tissue transplantation, when immunological conflict is minimized, depends on the vascularization process. This process is very complex and requires time. During this time, transplanted tissue often has difficulty obtaining oxygen and nutrients. These factors have a profound influence on the survival of the transplanted tissue, especially if the tissue has poor angiogenic properties. For new vessel formation, endothelial cells from existing recipient microvessels must proliferate and migrate through the extracellular matrix into the transplanted tissue. However, if transplanted tissue is mixed with recipient endothelial cells or co-administered, vascularization and acceptance of auto-, allo-, and heterotransplants is enhanced. In addition, recipient endothelial cells can be used to enhance vascularization of three-dimensional tissue-engineered constructs.

This application claims priority under 35 U.S.C. 119(e) to provisional application No. 60/316,441 filed Aug. 31, 2001 the contents of which are incorporated herein by reference.

### FIELD OF THE INVENTION

This invention relates to the vascularization of transplants. More particularly, it relates to the use of recipient endothelial cells to enhance vascularization of tissue and tissue-engineered construct transplants. Recipient endothelial cells can be used in combination with donor's tissues, as well as with allo- and hetero-transplants, and tissue-engineered constructs for better transplant vascularization.

### BACKGROUND OF THE INVENTION

Transplantation of organs, tissues, and tissue-engineered constructs is now commonplace in the treatment of a variety of medical conditions. For example, organs such as hearts and kidneys are now routinely transplanted in order to replace diseased organs. In addition, bone marrow transplantation is commonly performed as a treatment for leukemia and other hematological diseases. Other tissues, such as skin, are transplanted for a variety of reasons. More recently, complex tissue-engineered constructs have been prepared from biological and synthetic matrices containing various growth factors, therapeutics and/or cells. Collectively, these types of treatments can be referred to as “tissue transplantation”.

Solid organ transplants, such as heart, liver and kidney, are revascularized immediately upon reperfusion after the transplantation procedure. In contrast, the introduction of tissues such as islet cells, as well as various 2- and 3-dimensional tissue-engineered constructs (i.e. non-organ transplants) depends on new vessel formation, or “angiogenesis”, to ensure that the transplanted tissue receives an adequate blood supply. Accordingly, the success of tissue transplantation, when immunological conflict is absent (e.g. with auto- and singenic-transplantation, or allo- and heterotransplantation with immunodepletion) is highly dependent on the angiogenesis process (J. Folkman and M. Klagsbrun, Science 235:442-447 (1987); and C. H. Blood and B. R. Zetter, Biochim. Biophys. Acta, 1032(1): 89-118 (1990). This process is very complex and requires a significant amount of time, especially when the angiogenic properties of the transplanted tissue are compromised. It is during this time when the transplant is very vulnerable, because of a tenuous oxygen and nutrient supply, particularly in the more central portions of the transplant which are less reachable by diffusion. Thus, the post-transplantation angiogenic process has a profound influence on the survival of transplants such as islets (A. M. Davalli et al., Diabetes 19:1161-1167 (1996)).

Various metabolic processes may also influence transplant survival. Because of this, transplantation of isolated pancreatic islets as a treatment modality for diabetes mellitus has had limited success (J. I. Stranger, et al., Transplantation Proceedings 27(6): 3251-3254 (1995)). It is well documented that insulin secretion from transplanted islets is delayed and diminished when compared with secretion from a normal or transplanted pancreas. It has been suggested that a primary reason for nonimmune islet transplantation failure and inadequate insulin secretion may be the result of angiogenic inefficiency. Because transplanted islets require approximately 7 to 30 or more days for revascularization, it was suggested that this prolonged period of ischemia may be responsible for inadequate long-term beta-cell performance. This is quite understandable, since the beta-cells are located in the central portion of the islet where revascularization would take place last.

Angiogenisis is also an extremely complex process. It begins with the local dissolution of the basement membrane of an existing microvessel under the influence of endothelial derived proteases (D. Moscatelli and D. B. Rifkin, Biochem. Biophys. Acta., 948:67-85 (1988); and R. Montesano, et. al., Cell, 62: 435-445 (1990)). This is followed by endothelial cell proliferation and migration through the extracellular matrix toward the angiogenic stimulus. Finally, there is alignment of the migrating cells and formation of tubular structures. Microvascular tubes anastamose, forming a new capillary network through which blood flow is established.

Angiogenesis is also dependent on a complex signaling process that consists of two sets of extracellular signals. First, there are soluble factors that influence endothelial cell growth and differentiation. A very important group of soluble factors includes the heparin binding molecules that are related to acidic and basic fibroblasts growth factors (FGFs), as well as endothelial cell growth factor (ECGF) (W. H. Burgess and T. Maciag, Annual Rev. Biochem. 58: 575-606 (1989)). Other soluble factors that affect angiogenesis include TGF-beta, which inhibits proliferation and enhances differentiation of endothelial cells in vitro (M. S. Pepper, et al., J. Cell Biol. 111: 743-755 (1990)); platelet-derived growth factor (PDGF), which is among the most potent stimuli for cell migration in many cell types (J. Yu, et al., Biochem. Biophys. Res. Comm., 282(3): 697-700 (2001)); hypoxia-inducible factor 1 alpha (HIF-1 alpha) (E. Laughner, et al., Molecular and Cellular Biology 21(12): 3995-4004 (2001)); IL-1 and TNF (J. A. M. Maier, et al., Science 249: 1570-1574 (1990)); angiogenin, certain prostaglandins, and other low molecular weight substances (J. Bauer, et al., J. Cellular Physiol. 153: 437-449 (1992)).

The second major set of signals that regulate angiogenesis come from the extracellular matrix (M. Klagsbrun, J. Cell. Biochem. 47: 199-200 (1991)). Endothelial cell surface receptors of the integrin superfamily recognize extracellular matrix proteins that trigger a signaling event (S. M. Albelda C. A. Buck, FASEB J. 4 2868-2880 (1990)). It is suggested that the role of integrins is to maintain adhesive contact with the matrix and thus permit cell locomotion. However, this interaction may actually be more complex (J. Bauer, et al., J. Cellular Physiol. 153: 437-449 (1992)).

There have been attempts to improve the vascularization of transplanted pancreatic islets using acidic fibroblast growth factor. When syngeneic rat pancreas islets were transplanted into a kidney in the presence of this growth factor, the result was that more capillaries served the beta-cell-containing islet medulla, and a greater number of beta cells produced insulin (J. I. Stranger, et al., Transplantation Proceedings 27(6): 3251-3254 (1995)).

### SUMMARY OF THE INVENTION

The present invention relates, in part, to a method of transplanting tissue into a recipient to enhance vascularization of the tissue comprising the steps of: (a) obtaining endothelial cells from the recipient; (b) preparing the tissue for transplantation into a transplantation site on or in the recipient; and (c) administering the endothelial cells and the tissue to the transplantation site.

The tissue can be from natural sources, or it may take the form of a tissue-engineered construct. In addition, the transplant may be autologous, allogenic or heterogenic in nature.

The endothelial cells can be derived from the recipient's microvasculature, such as that found in adipose tissue or dermal microvascular beds.

In addition, the transplant may be contacted with the endothelial cells prior to implantation, or the transplant and the endothelial cells may be administered simultaneously.

In one embodiment, the present invention relates to a tissue-engineered construct for implantation into a human recipient comprising a tissue from an allogenic donor and endothelial cells from the recipient. The construct may contain, for example, cells from the pancreas, liver or kidney cells, along with a natural or synthetic cell scaffold, in addition to the recipient endothelial cells.

In another embodiment, the present invention relates to an islet for implantation into a human recipient comprising an allogenic islet and recipient endothelial cells infused therein.

In yet another embodiment of the present invention, there is provided a method for treating a human recipient with diabetes comprising transplanting an allogenic islet and recipient endothelial cells into a transplantation site of the recipient.

Other aspects of the present invention are described throughout the specification.

### DETAILED DESCRIPTION OF THE INVENTION

The present invention relates to the enhanced vascularization of non-organ transplants using recipient endothelial cells.

The Transplant

The present invention relates to tissue transplants as opposed to organ transplants. Tissue transplants may include, inter alia, bone, skin, connective tissue, heart tissue (including heart valves), vascular tissue and corneas. Organ transplants, on the other hand, include transplantation of whole organs such as the liver, kidneys, heart, lungs and pancreas. Unlike organ transplants that are performed less often in a fewer number of selected hospitals, tissue transplants are performed routinely at the majority of hospitals. In addition, there are important differences between the recovery of organs and tissues. Organs are recovered intact soon after death and require no processing before use. Tissue, on the other hand, can be recovered up to 24 hours after death and can be preserved through processes like freeze-drying and cryopreservation. Another more important difference in the context of the present invention is that organs do not require revascularization after transplantation, since their original vascular system remains largely in-tact, whereas tissues and tissue-eingineered constructs do.

In the practice of the present invention, the transplant may comprise tissue that is of natural origin, such as skin or bone marrow, or it may be cultured for purposes of transplantation. In addition, the transplant may comprise tissue-engineered constructs that are composed generally of a biological or synthetic matrix containing cells, which may also include various therapeutic agents and growth factors. Such tissue-engineered constructs may consist of donor cells and recipient endothelial cells, and may be used for the repair of many tissues and organs. In addition, in vitro constructs may be made from autologous adult stem cells and endothelial cells. These constructs would not be rejected, and would be expected to have enhanced vascularization after transplantation.

Skin transplants, more often called grafts, usually involve transplantation of a subject's own skin from one part of the body to another part of the body where the skin has sustained damage to the regenerative layers. This is called an “autograft”. Recent advances in cell culture techniques have contributed further to the success of skin transplants. It is now possible to remove a small section of skin from a burn victim, and grow it under controlled laboratory conditions. From an initial small sample, large sheets of epidermis have been grown and used to cover burn areas.

In contrast to autografts, “allografts” are transplants between individuals of the same species, and “heterogenic transplants” are transplants from different species. These transplants are complicated because of immunologic differences between donors and recipients that may result in rejection. However, the risk of rejection can be minimized by using various techniques to select donor tissues with enhanced compatibility, as well as the use of immunosuppressants, such as cyclosporine, to minimize the effects of the immune response following transplantation. However, the use of the immunosuppressants must be balanced against the risk of allowing the recipient to be vulnerable to pathogens, which could take full advantage of a compromised immune system.

Synthetic tissue-engineered constructs are also now used to transplant cells and tissues to treat a variety of different medical conditions. Tissue engineering involves the development of synthetic materials or devices that are capable of specific interactions with biological tissues. The constructs combine these materials with living cells to yield functional tissue equivalents. Such systems are useful for tissue replacement where there is a limited availability of donor organs or where, in some cases, (e.g., nerves) natural replacements are not readily. As used herein, the term “tissue-engineered constructs” includes any combination of naturally derived or synthetically grown tissue or cells, along with a natural or synthetic scaffold that provides structural integrity to the construct.

Tissue engineering involves a number of different disciplines, such as biomaterial engineering, drug delivery, recombinant DNA techniques, biodegradable polymers, bioreactors, stem cell isolation, cell encapsulation and immobilization, and the production of 2 dimensional and 3 dimensional scaffolds for cells.

Porous biodegradable biomaterial scaffolds are required for the 3 dimensional growth of cells to form the tissue engineering constructs. There are several techniques to obtain porosity for the scaffolds. Of these methods, fiber bonding, solvent casting/particulate leaching, gas foaming/particulate leaching and liquid-liquid phase separation produce large, interconnected pores to facilitate cell seeding and migration. The fiber bonding, solvent casting/particulate leaching and gas foaming/particulate leaching methods exhibit good biocompatibility, making these techniques especially promising for future use in tissue-engineered cell-polymer constructs.

Generally, the pores must be a size range that permits infiltration of a variety of different cells to grow within the scaffolds. In addition, depending on the size and shape of the construct, the scaffold must be biodegradable or porous enough to permit infiltration of endothelial cells and eventual angiogenesis.

In one aspect of the present invention, the tissue transplant is a pancreatic islet that is transplanted for the purpose of treating diabetes. There are two types of diabetes. Type I, which is the early onset form of diabetes, is characterized by immune-mediated destruction of the pancreatic islets. Patients with Type I diabetes become dependent on insulin for survival. In contrast, Type II diabetes is characterized by insulin resistance due to a lack of effective interaction between insulin and target cells. This type of diabetes usually occurs later in life and may or may not require insulin therapy.

There are many problems associated with the prolonged use of insulin to treat Type I diabetes, such as blindness, renal failure, neuropathy and arteriosclerosis. Although many advances have been made to more closely mimic normal insulin production, these problems arise from the inability to tightly balance blood glucose concentrations. Accordingly, many physicians have turned to pancreatic organ transplant, which is usually performed together with a kidney transplant, to attempt to preserve normal body functions of Type I diabetes patients. However, in many patients, the risk of such an invasive treatment is often prohibitive.

More recently, researchers have developed various means of isolating and transplanting the islets of Langerhans (“islets”) from the pancreas separately. Islets are made up of two types of cells: the alpha cells, which make glucagon, a hormone that raises the level of glucose (sugar) in the blood, and the beta cells, which make insulin. Within the human pancreas organ there are about 1-1.5 million islets of Langerhans. The islets make up about 2% of the mass of the pancreas, and Each islet contains between 2,000 and 10,000 cells.

In addition to transplantation of in-tact islets, many attempts have been made to transplant tissue-eingineered constructs containing beta-cells. However, just as with in-tact islets, it is important to promote microvascularization of the construct to enable the insulin secreted from the beta-cells to enter the general circulation, and also to provide the beta-cells with a source of oxygen and other nutrients.

The Endothelial Cells

The present invention relates to the discovery that combining tissue transplants with recipient endothelial cells, either before or during transplantation, promotes post-transplantation angiogenesis. In order to avoid immunologic rejection, recipient endothelial cells are used in the practice of the present invention as opposed to endothelial cells from other sources. Various means exist for avoiding immunologic rejection of the transplant itself, and by using recipient endothelial cells, a further source of immunologic incompatibility is avoided.

Endothelial cells from different sources exhibit varying mitogenic responses to cytokines. In general, the responses of endothelial cells from the microvasculature are superior. For example, human umbilical vein endothelial cells (HUVEC) and microvascular endothelial cells (MIEC) differ in their proliferative response to vascular endothelial growth factors (VEGFs), basic fibroblast growth factor (FGF-2), and placental growth factors (PlGFs). It has been shown that microvascular endothelial cells respond stronger to all three growth factors and are thus preferred over other sources (I. Lang, et al., Cell Proliferation, 34(3): 143-155 (2001).

Microvessels can be derived from, e.g., human dermal microvascular beds, umbilical veins, adipose tissue, and other tissues that are readily obtainable from the recipient and adequately vascularized. The recipient endothelial cells are used not only for improved vascularization of tissue transplants, but also for faster vascularization and better acceptance of tissue-engineered construct transplants. Inducing the growth of blood vessels is also key to sustaining many tissue-engineered constructs, particularly those containing cells from organs such as the pancreas, liver, and kidney, which require a large blood supply (R, S. Langer and J. P. Vacanti, Scientific American 280(4):87-89 (1999)). There have been attempts to stimulate new blood vessel formation in bioartificial tissues grown in the laboratory by coating the polymer scaffolding supporting the tissue with growth factors that trigger blood vessel formation (J. Mooney and A. G. Mikos, Scientific American, 280(4): 60-65 (1999)). However, these attempts must rely on infiltration of the recipient's endothelial cells after transplantation.

After harvesting the recipient endothelial cells, they may be cultured using known methods prior to use. For example, microvascular endothelial cells from humans and animals have been harvested from a variety of different sources and cultured (Petzelbauer et al., J. Immunol. 151: 5062-5072.)

Transplantation with Recipient Endothelial Cells

As used herein, the phrase “transplanting tissue” refers to both the transplantation of tissue from culture or from natural sources, as well as transplantation of tissue-engineered constructs that include tissue or cells. The transplant can be pretreated with recipient endothelial cells immediately prior to transplantation, transplanted simultaneously with the endothelial cells or, especially with tissue-engineered constructs, the transplant can be cultured with endothelial cells to enhance infiltration into the transplant prior to transplantation.

In most cases, it is sufficient to pretreat the transplant immediately before transplantation or to simultaneously administer endothelial cells to the site of transplantation. In any event, it is necessary to administer the endothelial cells in such a manner that they enhance vascularization of the transplant (i.e. it occurs faster and to a greater extent than if the recipient's endogenous endothelial cells were permitted to infuse the transplant.) However, it should be pointed out that the present invention does not intend transplantation of preformed vascular beds or other vascular structures, either separately or within the transplant, which would be time consuming and impractical using recipient endothelial cells.

Optional Embodiments

Various optional constituents, such as immunosuppressive agents, growth factors and other substances, can also be included with the endothelial cells and/or the transplant. Such constituents include, inter alia, extracellular matrix proteins such as collagen and fibronectin; integrins; growth factors such as tissue growth factors, etc. In particular, angiogenic factors can be administered along with the transplant, which include basic fibroblast growth factor, acidic fibroblast growth factor, endothelial cell growth factor, angiogenin, and transforming growth factors alpha and beta. Other optional transplant constituents are discussed in the background of invention.

### EXAMPLE

Six-week old nude BALB/c mice were used for this study. The cells of a Syrian hamster pancreatic beta cell line HIT-T15 (CRL-1777 from ATCC) were also used (Santerre, R. F., et al., Proc. Natl. Acad. Sci. USA, 78:4339-4343 (1981)). Cells were grown in Kaighn's modification of Ham's F12 medium with 10% horse serum and 3% fetal bovine serum. Nude BALB/c mice microvascular endothelial cells were obtained from lung vessels using collagenase injection in the microvascular circulation. The cells were propagated in EGM medium (Clonetic) with bovine brain extract-BBE (Clonetic). Cell cultures were grown in a 5% CO₂ in air atmosphere. Syrian hamster microvascular endothelial cells were similarly obtained and grown.

There were three groups of mice. The mice in the first group (N=10) were injected subcutaneously with 2.2×10⁶ HIT-T15 cells only. This group represents a control transplantation with no endothelial cells. The mice in the second group (N=12) were injected subcutaneously with 2×10⁶ HIT-T15 cells with 2×10⁵ hamster endothelial cells. This group represents a control transplant with donor endothelial cells. The mice in the third group (N=12) were injected subcutaneously with 2×10⁶ HIT-T15 cells with 2×10⁵ nude BALB/c mice endothelial cells. This group represents a test study to determine the effectiveness of transplantation of islet cells and endothelial cells.

Transplants were removed after 60 days and fixed with 10% neutral formaldehyde. Specimens were sectioned into 7 millimicron slices, and stained by hematoxylin-eosin. The number of vessels in the transplants was estimated by determining the mean number of vessels per 10 high power fields of vision on each of 5 slices (magnification 10×250).

In the first group injected with hamster pancreatic beta-cells alone, the transplants exhibited growth after 48.2±3.7 days. The mean number of vessels in these transplants was 7.6±0.9. This indicates that the control group is capable of supporting growth of beta-cells after transplantation.

In the second group injected with hamster pancreatic beta-cells and hamster endothelial cells, the transplants exhibited growth after 49.3±3.9 days. The mean number of vessels in these transplants was 7.2±1.5. This indicates that the presence of donor endothelial cells did not significantly alter the density of new vessel formation and acceptance of the tissue transplant.

In the third group injected with hamster pancreatic beta-cells and mice (representative or recipient) endothelial cells, the transplant exhibited growth after only 5±1.2 days. The mean number of vessels in these transplants was 40.7±4.0, and the vessels were larger in size than in the transplants of the two other groups. This indicates that mice endothelial cells stimulate the vascularization and growth rate of transplants.

The data from this experiment demonstrate that only recipient endothelial cells can stimulate vessel formation in tissue transplants, but non-recipient endothelial cells do not posses this ability.

The example set forth above is provided to give those of ordinary skill in the art with a complete disclosure and description of how to make and use the preferred embodiments of the compositions, and is not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

We claim:

1. A method of transplanting tissue into a recipient to enhance vascularization of the tissue comprising the steps of:
(a) obtaining endothelial cells from the recipient;
(b) preparing the tissue for transplantation into a transplantation site on or in the recipient to obtain a transplant; and
(c) administering the endothelial cells and the tissue to the transplantation site.

2. The method of claim 1, wherein the tissue is from natural sources.

3. The method of claim 1, wherein the tissue is a tissue-engineered construct.

4. The method of claim 1, wherein the transplant is an autologous transplant.

5. The method of claim 1, wherein the transplant is an allogenic transplant.

6. The method of claim 1, wherein the transplant is a heterogenic transplant.

7. The method of claim 1, wherein the endothelial cells are obtained from the recipient's microvasculature.

8. The method of claim 1, wherein the endothelial cells are obtained from adipose tissue or dermal microvascular beds.

9. The method of claim 1, wherein the tissue is contacted with the endothelial cells prior to transplantation.

10. The method of claim 1, wherein the tissue and the endothelial cells are administered to the transplantation site simultaneously.

11. A tissue-engineered construct for implantation into a human recipient comprising a tissue from an allogenic donor and endothelial cells from the recipient.

12. An islet for implantation into a human recipient comprising an allogenic islet and recipient endothelial cells infused therein.

13. A method for treating a human recipient with diabetes comprising transplanting an allogenic islet and recipient endothelial cells into a transplantation site of the recipient.

14. The tissue-engineered construct according to claim 11, wherein the tissue further comprises pancreatic, liver or kidney cells, a natural or synthetic cell scaffold, and recipient endothelial cells.
